(19) United States
(12) Patent Application Publication
(10) Pub. No.: US 2010/0190860 A1
(43) Pub. Date: Jul. 29, 2010

(54) METHODS FOR SELECTIVELY ENHANCING ANTINOCICEPTIVE POTENCY OF LOCAL ANESTHETICS

(75) Inventors: Peter Gerner, Weston, MA (US); Ging Kou Wang, Brookline, MA (US)

Correspondence Address: FISH & RICHARDSON PC, P.O. BOX 1022, MINNEAPOLIS, MN, 55440-1022, US

(73) Assignee: The Brigham and Women's Hospital, Inc., Boston, MA (US)

(21) Appl. No.: 12/684,778

(22) Filed: Jan. 8, 2010

Related U.S. Application Data
(60) Provisional application No. 61/143,435, filed on Jan. 9, 2009.

(51) Int. Cl. A61K 31/165; A61K 31/14; A61P 23/02

(52) U.S. Cl. 514/627; 514/643

(57) ABSTRACT

This document features methods related to selectively inducing nociceptor blockade. For example, methods of treating pain by administering antinociceptor agents are provided.

### CROSS-REFERENCE TO RELATED APPLICATION

This application is based on, and claims the benefit of, U.S. Provisional Application No. 61/143,435 filed on Jan. 9, 2009, which is incorporated by reference herein in its entirety.

### FEDERALLY SPONSORED RESEARCH OR DEVELOPMENT

The U.S. Government has certain rights in this invention pursuant to Grant Nos. GM64051 and GM48090 awarded by the National Institutes of Health.

### TECHNICAL FIELD

This document features methods related to selectively inducing nociceptor blockade. For example, this document provides methods of treating pain by administering antinociceptive agents.

### BACKGROUND

Nociception is the neural process that culminates in the perception of a painful stimulus. Nociception is triggered in response to noxious stimuli received by sensory receptors known as nociceptors. The perception of pain can be alleviated by blocking the activity of such sensory receptors. Antinociceptive agents comprise a large class of drugs that are used to alleviate pain. They include compounds such as local anesthetics, steroids, barbiturates, and opioids. In addition to blocking voltage-gated sodium channels in sensory nerve fibers, local anesthetics (LAs) also block sodium channels in motor and sympathetic fibers. Therefore, complete pain relief is generally only accomplished with concomitant low-threshold sensory afferent blockade, sympathetic blockade causing low blood pressure, and motor blockade causing immobility. Despite advances in developing methods of sensory-selective analgesia, there remains a need to optimize sensory selectivity of local anesthetics as a means of expanding their clinical utility.

### SUMMARY

This document describes methods for selectively blocking nociceptive receptors by sequentially or simultaneously administering one or more local anesthetics and a transient receptor potential cation channel, subfamily V, member 1 (TRPV1) receptor agonist without affecting motor function. As described herein, methods of treating pain by administering local anesthetics and TRPV1 receptor agonists are provided. Such methods can have substantial value for clinical use.

In one aspect, this document features a method for treating pain in a subject. The method can comprise administering to the subject effective amounts of (i) one or more membrane-permeant local anesthetic agents and (ii) a transient receptor potential cation channel, subfamily V, member 1 (TRPV1) receptor agonist. In some cases, the subject can be about to undergo a surgical or other painful procedure. The one or more local anesthetic agents can be administered prior to administering said TRPV1 receptor agonist. The one or more membrane-permeant local anesthetics can be a tricyclic antidepressant. The tricyclic antidepressant can be selected from the group consisting of amitriptyline, amoxapine, clomipramine, desipramine, doxepin, imipramine, nortriptyline, protriptyline, and trimipramine, or an analog or derivative thereof. The one or more membrane-permeant local anesthetic agents can be selected from the group consisting of N-methyl amitriptyline, amitriptyline, bupivacaine, lidocaine, N-methyl doxepine, and N-methyl nortriptyline, or an analog or derivative thereof. The TRPV1 receptor agonist can be an endovanilloid; an endocannabinoid; an anandamide; or a lipoxygenase metabolite of arachidonic acid. The endovanilloid can be capsaicin. The mode of administration can be intramuscular, subcutaneous, perineural, neuraxial, or transdermal administration. The administration of the one or more membrane-permeant local anesthetics and the administration of said TRPV1 receptor agonist can be sequential or substantially simultaneous. The administration of one or more membrane-permeant local anesthetics and the administration of the TRPV1 receptor agonist can be separated by a predetermined interval of time. The one or more membrane-permeant local anesthetics can be N-methyl-amitriptyline and the TRPV1 receptor agonist can be capsaicin. The N-methyl-amitriptyline and the capsaicin can be substantially simultaneously administered by continuous infusion. The N-methyl-amitriptyline and the capsaicin can be substantially simultaneously administered as a bolus.

In another aspect, this document features a composition. The composition can comprise an analgesic amount of a TRPV1 receptor agonist and an amount of N-methyl-amitriptyline effective to treat pain in a subject administered the composition.

In another aspect, this document features a method for treating pain in a subject. The method can comprise administering to the subject a therapeutically effective amount of a composition comprising an analgesic amount of a TRPV1 receptor agonist and an amount of N-methyl-amitriptyline effective to treat pain in a subject administered the composition.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 demonstrates sciatic nerve block following injection of 0.2 mL of N-methyl amitriptyline (NMA) at 0.125%, either alone or in combination with 0.05% capsaicin (simultaneously applied/mixed or 10 minutes following the initial injection), or followed by vehicle only. N=8 rats per group. Data are presented as sum score of blockade for motor and nociceptive function. The center line is the median, the lower and upper boundaries are the 25th and 75th percentiles, and the error bars are the 5th and 95th percentiles. ** indicates p<0.01 for nociceptive block of NMA combined with capsaicin as compared to NMA injection alone.

FIG. 2 demonstrates sciatic nerve block following injection of 0.2 mL of amitriptyline (AMI) at 0.125%, either alone or in combination with 0.05% capsaicin (simultaneously applied/mixed or 10 minutes later), or followed by vehicle only. N=8 rats per group. Data are presented as sum score of blockade for motor or nociceptive function. The center line is the median, the lower and upper boundaries are the 25th and 75th percentiles, and the error bars are the 5th and 95th percentiles. * indicates p<0.05/3 (0.0167), *** indicates p<0.001 for nociceptive block for respective amitriptyline/capsaicin or amitriptyline/capsaicin vehicle combinations vs. amitriptyline alone. # indicates p<0.05/3 (0.0167), ## indicates p<0.01, ### indicates p<0.001 for motor block for amitriptyline/capsaicin or amitriptyline/capsaicin vehicle combinations vs. amitriptyline alone.

FIG. 3 demonstrates sciatic nerve block following injection of 0.2 mL of bupivacaine at 0.25%, either alone or in combination with 0.05% capsaicin (simultaneously applied/mixed or 10 minutes later), or followed by vehicle only. N=8 rats per group. Data are presented as sum score of blockade for motor or nociceptive function. The center line is the median, the lower and upper boundaries are the 25th and 75th percentiles, and the error bars are the 5th and 95th percentiles. *** indicates p<0.001 for nociceptive block for respective bupivacaine/capsaicin or bupivacaine/capsaicin vehicle combinations vs. bupivacaine alone. # indicates p<0.05/3 (0.0167) for motor block for bupivacaine/capsaicin or bupivacaine/capsaicin vehicle combinations vs. bupivacaine alone.

FIG. 4 demonstrates sciatic nerve block following injection of 0.2 mL of lidocaine at 2%, either alone or in combination with 0.05% capsaicin (simultaneously applied/mixed or 10 minutes later), or followed by vehicle only. N=8 rats per group. Data are presented as sum score of blockade for motor or nociceptive function. The center line is the median, the lower and upper boundaries are the 25th and 75th percentiles, and the error bars are the 5th and 95th percentiles. * indicates p<0.05/3 (0.0167), ** indicates p<0.01 for nociceptive block for respective lidocaine/capsaicin or lidocaine/capsaicin vehicle combinations vs. lidocaine alone. # indicates p<0.05/3 (0.0167) for motor block for lidocaine/capsaicin vehicle combination vs. lidocaine alone.

FIGS. 5A-B contain a dose-response curve for N-methyl amitriptyline (NMA), either alone or in combination with 0.05% capsaicin (applied 10 minutes later). N=8 rats per group. Data are presented as sum score of blockade for (A) motor or (B) nociceptive function. The center line is the median, the lower and upper boundaries are the 25th and 75th percentiles, and the error bars are the 5th and 95th percentiles.

FIGS. 6A-B contain a dose-response curve for amitriptyline (AMI), either alone or in combination with 0.05% capsaicin (applied 10 min later). N=8 rats per group. Data are presented as sum score of blockade for (A) motor or (B) nociceptive function. The center line is the median, the lower and upper boundaries are the 25th and 75th percentiles, and the error bars are the 5th and 95th percentiles.

FIG. 7 is a graph demonstrating inhibition of the cutaneous trunci muscle reflex following transdermal administration of amitriptyline in combination with capsaicin or amitriptyline alone.

### DETAILED DESCRIPTION

The present invention is based at least in part on the discovery that local anesthetics are capable of producing a pronounced and long-lasting predominantly nociceptor-selective nerve blockade when used with capsaicin, a TRPV1 receptor agonist. Based at least in part on these discoveries are methods for substantially selectively blocking nociceptive receptors and not or only mildly affecting motor function by sequentially or simultaneously administering one or more local anesthetics and a TRPV1 receptor agonist. As described herein, methods of alleviating and treating pain using such methods of administering analgesic compounds are provided.

As used herein, the terms “treat” and “treating” mean decreasing, suppressing attenuating, diminishing, arresting, preventing (e.g., for patients about to undergo surgical or other painful procedures), lessening the severity of, or improving symptoms (e.g., pain) which can be associated with a disease, disorder, or medical procedure.

As used herein, the term “effective amount” refers to an amount which, when administered in a proper dosing regimen, is sufficient to reduce or ameliorate the severity, duration, or progression of the disorder being treated, prevent the advancement of the disorder being treated, cause the regression of the disorder being treated, enhance or improve the prophylactic or therapeutic effect(s) of another therapy, or to promote a healthy weight. Effective amounts will vary, as recognized by those skilled in the art, depending on the severity of the disorder, the route of administration, excipient usage, the possibility of co-administration with other therapeutic or prophylactic treatments, and the judgment of the treating physician, clinician, or other healthcare provider.

As used herein, the term “nociception” refers to the neural process of perceiving pain that is triggered by the reception of noxious stimuli by somatic and visceral free nerve endings called nociceptors.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio.

This document provides methods of selectively or predominantly blocking nociception, and almost completely sparing motor function, by administering a therapeutically effective amount of one or more local anesthetics and an agonist of the transient receptor potential cation channel, subfamily V, member 1 (TRPV1) (e.g., capsaicin, resiniferatoxin, N-arachidonoyl-dopamine, anandamide, or N-oleoyldopamine). In some cases, such compounds can be administered in combination with a pharmaceutically acceptable carrier. This document also provides methods for treating pain in a subject suffering therefrom. The methods provided herein can further include identifying a subject in need of treatment for pain.

In the methods provided herein, an effective amount of one or more local anesthetic agents and a TRPV1 receptor agonist can be sequentially administered to a subject to selectively or predominantly block nociception, e.g., the local anesthetic can be administered before the TRPV1 receptor agonist. For example, amitriptyline can be administered to a subject, and subsequently a TRPV1 receptor agonist such as capsaicin can be administered after a predetermined interval of time. In some cases, depending on drug and dosage, the interval of time can be about zero, one, or more minutes (e.g., about 1, 2, 3, 5, 10, 15, 30, 45, or more) or hours (e.g., about 1, 1.5, 2, 4, 6, or more) between administrations of the analgesic agents. The analgesic agents can be administered as a bolus, or can be administered continuously for an interval of time, up to several days.

In some cases, a composition comprising an effective amount of one or more local anesthetics and a TRPV1 receptor agonist combined can be administered substantially simultaneously to a subject to selectively or predominantly block nociception. For example, a composition comprising N-methyl-amitriptyline and capsaicin can be administered to a subject. In some cases, N-methyl-amitriptyline and capsaicin can be administered substantially simultaneously for a time and under conditions effective to selectively or predominantly induce nociceptor analgesia. The analgesic agents can be administered substantially simultaneously as a bolus, or can be administered continuously for an interval of time, up to several days. For example, bupivacaine and capsaicin can be administered substantially simultaneously and continuously by infusion for an interval of time.

### Local Anesthetics

As described herein, compositions for use in the methods provided herein can include local anesthetics. Local anesthetics (LA) are compounds that produce reversible local anesthesia and a loss of nociception. Any appropriate local anesthetic can be used. For example, ionizable local anesthetics (e.g., non-clinical LA amitriptyline) and clinically-used local anesthetics (e.g., bupivacaine and lidocaine) can be used. In some cases, a local anesthetic can be, for example, a tricyclic antidepressant. See U.S. Pat. No. 7,074,961, incorporated herein by reference in its entirety. Examples of tricyclic antidepressants include, without limitation, amitriptyline, amoxapine, clomipramine, desipramine, doxepin, imipramine, nortriptyline, protriptyline, trimipramine, or an analog or derivative thereof. Analogs and derivatives of local anesthetics can include quaternary and tertiary analogs. For example, quaternary amitriptyline analogs include but are not limited to: N-phenyl-propyl amitriptyline bromide, N-phenyl-ethyl amitriptyline bromide, or N-phenyl-methyl amitriptyline bromide. Tertiary amitriptyline analogs include but are not limited to: N-phenyl-propyl nortriptyline bromide, N-phenyl-ethyl nortriptyline bromide, or N-phenyl-methyl nortriptyline bromide. Other local anesthetics can include benzocaine, chloroprocaine, cocaine, cyclomethycaine, dimethocaine/larocaine, propoxycaine, procaine/novocaine, proparacaine, tetracaine/amethocaine, articaine, carticaine, cinchocaine/dibucaine, etidocaine, levobupivacaine, mepivacaine, piperocaine, prilocalne, ropivacaine, trimecaine. In some cases, positively-charged, neutral and/or membrane-permeant local anesthetics can be used. Less membrane-permeant LAs are less able to block sodium channel from the cytoplasmic side and may exhibit slow onset of nociceptive blockade or no nociceptive blockade. See Binshtok et al., Nature 449:607-10 (2007); Strichartz, J. Gen. Physiol. 62:37-57 (1973). Membrane-permeant LAs can include, without limitation, N-methyl amitriptyline, N-methyl doxepine, N-methyl nortriptyline, and any other N-alkyl tricyclicantidepressant. In some embodiments, membrane-permeant local anesthetics do not include QX314 or bupivacaine.

### TRPV1 Receptor Agonists

A TRPV1 receptor agonist can be, for example, an endovanilloid such as capsaicin, resiniferatoxin (a naturally-occurring analog of capsaicin), or N-oleoyldopamine; an endocannabinoid such as N-arachidonoyl-dopamine; anandamide (an endogenous TRPV1 ligand); or a lipoxygenase metabolite of arachidonic acid such as 12-(S)-hydroperoxyeicosatetraenoic acid (12-(S)-HPETE), 15-(S)-hydroperoxyeicosatetraenoic acid (15-(S)-HPETE), lidocaine, and leukotriene B4 (LTB₄). Capsaicin (8-methyl-N-vanillyl-6-nonenamide) is produced as a secondary metabolite by chili peppers, which are plants belonging to the genus Capsicum. Capsaicin selectively binds to TRPV1, a member of the superfamily of transient release potential ion channels which is expressed peripherally in primary afferent nociceptors of the peripheral nervous system.

### Administration

Any suitable mode of administration can be used. For example, administration can be intramuscular, subcutaneous, topical, transdermal, transmucosal, perineural, or neuraxial administration. For intramuscular, or subcutaneous administration, analgesic compounds may combined with a pharmaceutically acceptable carrier such as a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. Other pharmaceutically acceptable carriers, adjuvants, and vehicles that may be used in the compositions described herein can include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat. Formulations may be present in unit or multi-dose containers such as prefilled syringes, sealed ampoules, or vials.

For topical administration to the epidermis, the analgesic compounds can be formulated as creams, gels, ointments or lotions or as transdermal patches. Such compositions can, for example, be formulated with an aqueous or oily base with the addition of suitable thickening, gelling, emulsifying, stabilizing, dispersing, suspending, and/or coloring agents. For transdermal administration, the compounds may be combined with skin penetration enhancers such as propylene glycol, polyethylene glycol, isopropanol, ethanol, oleic acid, N-methylpyrrolidone, which increase the permeability of the skin to the compounds, and permit the compounds to penetrate through the skin and into the bloodstream.

As described herein, the analgesic compounds are administered to a subject at a dosage sufficient to achieve the desired therapeutic effect (e.g., treating pain). In general, therapeutically effective dosages may be determined by either in vitro or in vivo methods. The interrelationship of dosages for animals and humans (based on milligrams per meter squared of body surface) is described in Freireich et al., Cancer Chemother. Rep. 50:219 (1966). Body surface area can be approximately determined from height and weight of the subject. See, e.g., Scientific Tables, Geigy Pharmaceuticals, Ardsley, N.Y., 1970, 537. Dosage values may vary according to factors such as the disease state, age, sex, and weight of the individual.

A therapeutically effective amount of a local anesthetic agent can range between about 1 milligram (mg) and about 400 mg (e.g., about 0.5, 1, 2, 3, 4, 5, 10, 20, 40, 50, 75, 80, 90, 100, 120, 140, 160, 180, 200, 220, 240, 260, 280, 300, 320, 340, 360, 380, or 400 mg). For example, an effective amount of the local anesthetic agent amitriptyline can be about 5 mg. In some cases, an effective amount of the local anesthetic agent lidocaine can be about 400 mg. In some cases, an effective amount of the local anesthetic agent bupivacaine can be about 3 mg. In some cases, an effective amount of the local anesthetic agent N-methyl-amitriptyline can be about 1 mg.

An effective amount of a TRPV1 receptor agonist can range between about 0.1 mg and about 5 mg (e.g., about 0.1, 0.2, 0.5, 0.8, 1, 1.5, 2, 3, 4, or 5 mg). For example, an effective amount of the TRPV1 receptor agonist capsaicin can be about 5 mg. In some cases, an effective amount of the TRPV1 receptor agonist resiniferatoxin can be about 0.1 mg.

The concentration of capsaicin used can vary according to the type and dosage of local anesthetic to be co-administered. In general, a higher concentration of capsaicin can be used when co-administered with a short-onset local anesthetic. The concentration of capsaicin can range between 0.05% to about 8-10%. In some cases, capsaicin is used at a concentration between about 0.05% and about 0.5%.

The effect of the methods provided herein on nociception can be monitored by subjective (e.g., observation or self-report) and/or objective indicators (e.g., measurable by a test or diagnostic method). Any suitable method of detecting and evaluating nociceptor blockade can be used. For example, a subject's response to analgesic agents administered as provided herein can be evaluated by detecting and/or scoring a flinch response, escape response, or agitation evoked by a transient, strong stimulus. In some cases, an apparatus for detecting such behavioral responses can be used. See, e.g., U.S. Pat. No. 6,996,429.

### EXAMPLES

The invention will be further described in the following examples, which do not limit the scope of the invention described in the claims.

### Example 1

### In Vivo Sciatic-Nerve Injections

Capsaicin, amitriptyline hydrochloride, bupivacaine hydrochloride, and lidocaine hydrochloride were purchased from Sigma Chemical Co., St. Louis, Mo. N-methyl amitriptyline, which was custom synthesized by Sigma Chemical Co., had greater than 99% purity by high-performance liquid chromatography and a molecular weight of 372.3. Capsaicin was freshly prepared with a solvent of 10% ethanol, 10% Tween® 80, and 80% normal saline (pH of the final solution was 6.6). All other drugs were freshly dissolved in 0.9% NaCl (pH ranged from 5.0 to 6.0). The pH was not adjusted because it is probably buffered quickly by the pH of the tissue fluid (7.4).

The animal experimental protocol was approved by the Standing Committee on Animals of Harvard Medical School, Boston, Mass. Male Sprague-Dawley rats were purchased from Charles River Laboratories, Inc. (Wilmington, Mass.) and kept in animal housing facilities with controlled relative humidity (20-30%), at room temperature (24° C.), in a 12-hour (6:00 a.m. to 6:00 p.m.) light-dark cycle. Rats were handled before the procedures to familiarize them with the experimental environment and to minimize stress-induced analgesia. At the time of injection, animals weighed 250 to 300 g.

The rats were assigned to treatments via block randomization with a block size of 8. All rats were anesthetized by inhalation of 1-2% of sevoflurane (Abbott Laboratory, North Chicago, Ill.) until no withdrawal to pinch of the leg occurred (by forceps). After induction of inhalation anesthesia, the drug in a volume of 200 μL was injected at the sciatic notch of the left hind limb with a 27-gauge needle connected to a tuberculin syringe: (1) N-methyl amitriptyline at 0.125%/3.4 mM; (2) amitriptyline at 0.125%/4.0 mM; (3) bupivacaine at 0.25%/7.3 mM; and (4) lidocaine at 2%/73.9 mM. All drugs were given alone or co-administered with capsaicin at 0.05%/1.6 mM. Co-administration of capsaicin was performed either 10 minutes after the first drug or simultaneously (e.g., mixed with the local anesthetic). The vehicle control group received local anesthetic followed by injection of vehicle 10 minutes later. Capsaicin was also injected alone as was normal saline. Each drug was injected was injected in a volume of 0.2 mL. The experimenter was blinded to the drug used, but was not blinded to the administration of a second drug 10 minutes later which was either capsaicin or vehicle alone.

### Example 2

### Neurobehavioral Examination

The effects of local anesthetics and capsaicin on motor function and nociception were evaluated. Rats were examined before injection for baseline functions, and at 10, 20, 30, 60, and 90 minutes and 2, 3, 6, 12, 24, and 36 hours after drug administration. Motor function was assayed by holding the rat upright with the control hind limb extended so that the distal metatarsus and toes of the target leg supported the animal's weight; the extensor postural thrust was recorded as the force (in grams) applied by each of the two hind limbs to a digital platform balance (Ohaus Lopro; Fisher Scientific, Florham Park, N.J.). The reduction in this force, representing reduced extensor-muscle contraction caused by motor block, was calculated as a percentage of the control force (pre-injection control value=145 to 165 g). The percent reduction in force was assigned a ‘range’ score: 0=no block (or baseline); 1=minimal block, force between the pre-injection control value of 100% and 50%; 2=moderate block, force between 50% of the pre-injection control value and 20 g (˜20 g represented the approximate weight of the flaccid limb); 3=complete block, force 20 g or less.

Nociception was evaluated by the nocifensive withdrawal reflex and vocalization to pinch of a skin fold over the lateral metatarsus (cutaneous pain) with a serrated forceps; the force and duration of this pinch was held as constant as possible. The extent of the nocifensive withdrawal reflex and vocalization were combined on a scale of 0 to 3 for each examination. Grading was as follows: 3=complete block, no nocifensive reaction or vocalization; 2=moderate block, vocalization accompanied by slow withdrawal and flexion of the leg; 1=minimal block, brisk flexion of the leg, with some sideways movement of the body or other escape response and loud vocalization; 0 indicates the baseline with no block and all nocifensive responses listed above.

Testing of nociception was limited to superficial nociceptive block (i.e., pinching of a skin fold at the lateral area of the dorsum of the paw) since pilot studies utilizing pinching of the fifth toe had revealed nonreproducible results, perhaps because the presumed preferential C-fiber block of various drug combinations does not block large motor (proprioceptive) fibers, which would allow the rat to sense the pressure of the forceps when the entire fifth toe is moved and pinched. However, firm pinch with a serrated forceps to an entire skin fold at the lateral aspect of the dorsum elicited a very robust (anti)nociceptive response.

For both nociceptive and motor assessment, the examination was repeated three times at each time point and reported as an average of the three exams. After sciatic nerve block, all rats in the treatment groups (n=8 per group) showed a functional loss of nociceptive and motor function of different degrees and durations that were completely reversed over time.

Because of the ordinal categorical nature of the block scores, an overall test for drug effect was obtained via generalized estimating equations for longitudinal ordinal data. A cumulative logistic ordinal model was fit with a linear and quadratic trend in time and time-by-group interaction. The group effect and group and time interaction effect were tested using contrast coefficients in generalized estimating equations analysis. The overall P value was calculated via PROC GENMOD (SAS 9.1; Cary, N.C.). In order to have an overall 5% type I error rate for each drug-mode, a p-value of less than 0.0167 was considered statistically significant (0.05/3=0.0167, because there are 3 sets of comparisons among each drug-mode).

### Example 3

### Results of Sequential and Simultaneous Injections

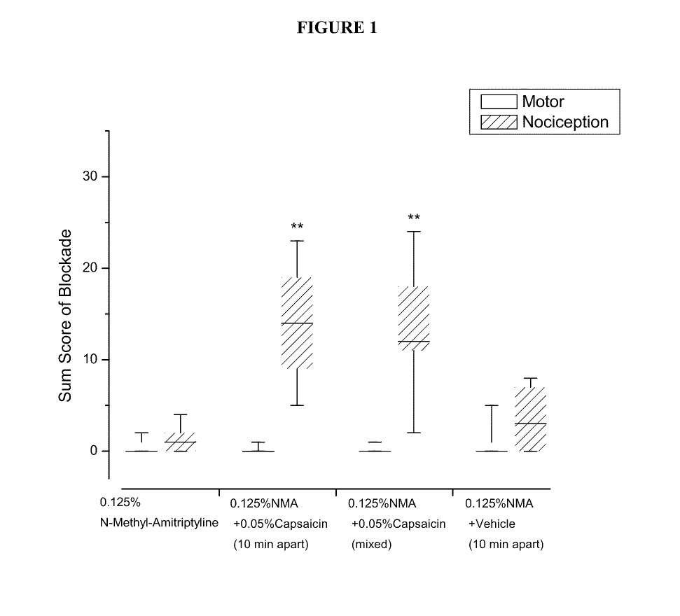

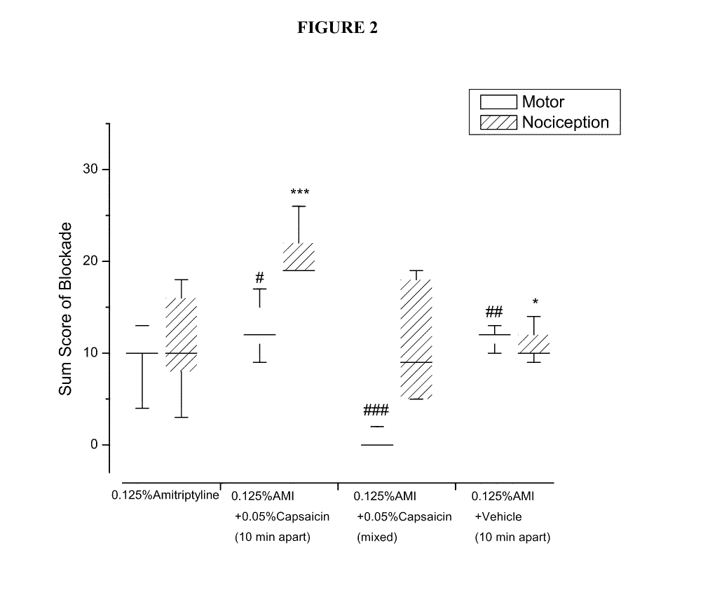

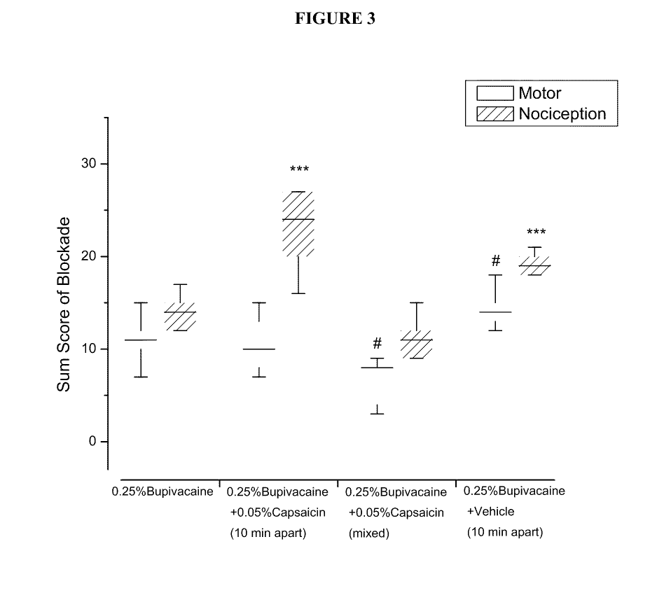

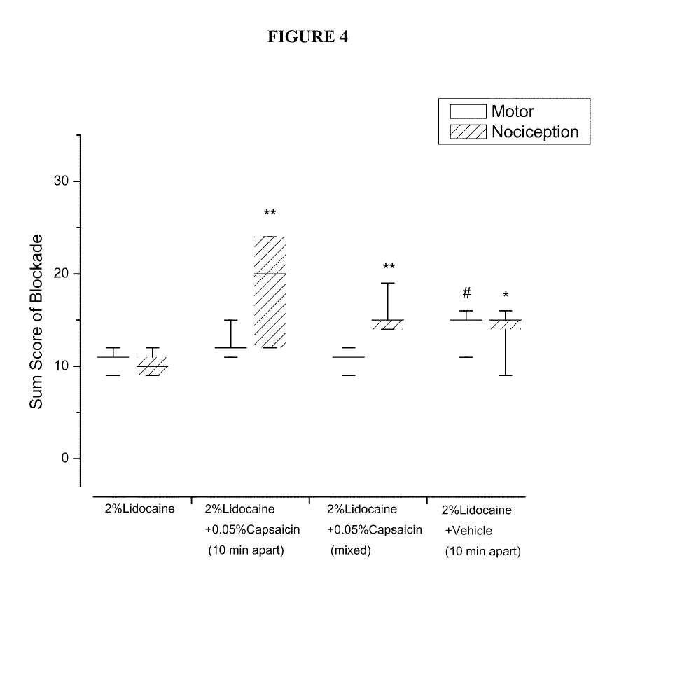

Overall, N-methyl amitriptyline (FIG. 1), amitriptyline (FIG. 2), bupivacaine (FIG. 3), and lidocaine (FIG. 4), with injection of capsaicin 10 minutes later, produced a predominantly nociceptive-specific blockade in this rat sciatic nerve block model. In contrast, simultaneous application of the local anesthetics with capsaicin did not produce a significant differential block, with the exception of N-methyl amitriptyline (Tables 1 and 2).

A. N-methyl Amitriptyline

For the 0.125% N-methyl amitriptyline solution, the duration of motor and nociceptive blockade was relatively brief (<1 hour) and incomplete when administered alone. When capsaicin 0.05% was added, either 10 minutes after the injection of N-methyl amitriptyline or injected simultaneously, a very prolonged sensory-selective block resulted (FIG. 1).

B. Amitriptyline

The duration of the motor block for 0.125% amitriptyline alone was similar to the duration of the nociceptive block, but addition of capsaicin produced a large differential block and significantly decreased the motor blockade when given simultaneously (FIG. 2).

C. Bupivacaine

Bupivacaine at a concentration of 0.25% was almost indistinguishable from 0.125% amitriptyline, when given alone or in combination with capsaicin. Injection of bupivacaine followed by capsaicin or vehicle 10 minutes later significantly increased the nociceptive blockade over motor blockade (FIG. 3).

D. Lidocaine

Lidocaine at 2% showed a complete but relatively short-lasting motor and nociceptive block when given alone, and the smallest differential block among all drug combinations when the lidocaine injection was followed by capsaicin (FIG. 4).

TABLE 1
Complete Recovery Times for Sequential and Simultaneous Injections

| | | | | Drug + 0.05% | | Drug + 0.05% | | | |
|---|---|---|---|---|---|---|---|---|---|
| | | | | Capsaicin | | Capsaicin | | Drug + Vehicle | |
| | | Drug Alone | | (10 min apart) | | (mixed) | | (10 min apart) | |
| | | Mean | SE | Mean | SE | Mean | SE | Mean | SE |
| 0.0625% N- | Motor | 0.12 | 0.08 | 0.06 | 0.06 | | | | |
| Methyl | Nociception | 0.00 | 0.00 | 0.13 | 0.08 | | | | |
| Amitriptyline | | | | | | | | | |
| 0.125% N- | Motor | 0.31 | 0.16 | 0.04 | 0.04 | 0.12 | 0.06 | 0.38 | 0.18 |
| Methyl | Nociception | 0.67 | 0.23 | 11.63 | 1.74 | 10.87 | 2.07 | 0.81 | 0.21 |
| Amitriptyline | | | | | | | | | |
| 0.25% N- | Motor | 1.44 | 0.32 | 1.81 | 0.21 | | | | |
| Methyl | Nociception | 3.63 | 0.18 | 33.00 | 1.13 | | | | |
| Amitriptyline | | | | | | | | | |
| 0.0625% | Motor | 0.44 | 0.15 | 1.38 | 0.08 | | | | |
| Amitriptyline | Nociception | 0.25 | 0.16 | 2.88 | 0.29 | | | | |
| 0.125% | Motor | 3.44 | 0.37 | 3.25 | 0.31 | 0.44 | 0.22 | 2.62 | 0.18 |
| Amitriptyline | Nociception | 4.56 | 1.04 | 17.25 | 0.75 | 2.69 | 0.69 | 2.13 | 0.21 |
| 0.25% | Motor | 9.13 | 1.33 | 2.31 | 0.63 | | | | |
| Amitriptyline | Nociception | 11.25 | 2.12 | 33.37 | 3.08 | | | | |
| 0.25% | Motor | 2.31 | 0.21 | 2.31 | 0.21 | 1.56 | 0.11 | 2.87 | 0.23 |
| Bupivacaine | Nociception | 2.50 | 1.89 | 23.14 | 3.32 | 1.63 | 0.13 | 4.75 | 0.25 |
| 2% | Motor | 1.13 | 0.08 | 1.69 | 0.09 | 1.50 | 0.13 | 2.00 | 0.16 |
| Lidocaine | Nociception | 1.25 | 0.09 | 7.62 | 1.47 | 3.00 | 0.27 | 2.12 | 0.23 |

Complete recovery times (in hours) for drug alone, drug combined with capsaicin (mixed or 10 minute apart), or drug followed by capsaicin vehicle.

TABLE 2
Pair-Wise Analysis of Group Effect and Group + Time Effect

| | | Drug alone vs. Drug + | | Drug alone vs. Drug + | | Drug alone vs. | |
|---|---|---|---|---|---|---|---|
| | | 0.05% Capsaicin | | 0.05% Capsaicin | | Drug + Vehicle | |
| | | (10 min apart) | | (mixed) | | (10 min apart) | |
| | | Chi- | | Chi- | | Chi- | |
| | | Square | | Square | | Square | |
| | Blockade | estimate | P value | estimate | P value | estimate | P value |
| 0.125% N- | Motor | x | x | x | x | x | x |
| Methyl | Nociception | 13.22 | 0.0013 | 11.09 | 0.0039 | 2.25 | 0.3239 |
| Amitriptyline | | | | | | | |
| 0.125% | Motor | 8.41 | 0.0149 | 14.37 | 0.0008 | 12.35 | 0.0021 |
| Amitriptyline | Nociception | 14.58 | 0.0007 | 7.05 | 0.0294 | 8.51 | 0.0142 |
| 0.25% | Motor | 3.28 | 0.1941 | 8.73 | 0.0127 | 8.57 | 0.0138 |
| Bupivacaine | Nociception | 14.15 | 0.0008 | 5.51 | 0.0635 | 17.70 | 0.0006 |
| 2% Lidocaine | Motor | 6.79 | 0.0336 | 3.05 | 0.2177 | 8.65 | 0.0132 |
| | Nociception | 10.91 | 0.0043 | 9.93 | 0.0070 | 8.43 | 0.0148 |

Pair-wise analysis of group effect and group and time effect for each drug given alone versus drug with capsaicin (10 minutes apart or simultaneous) or capsaicin vehicle, showing chi-square and p-value obtained using generalized estimating equations (GEE).

### Example 4

### Dose-Response Analysis

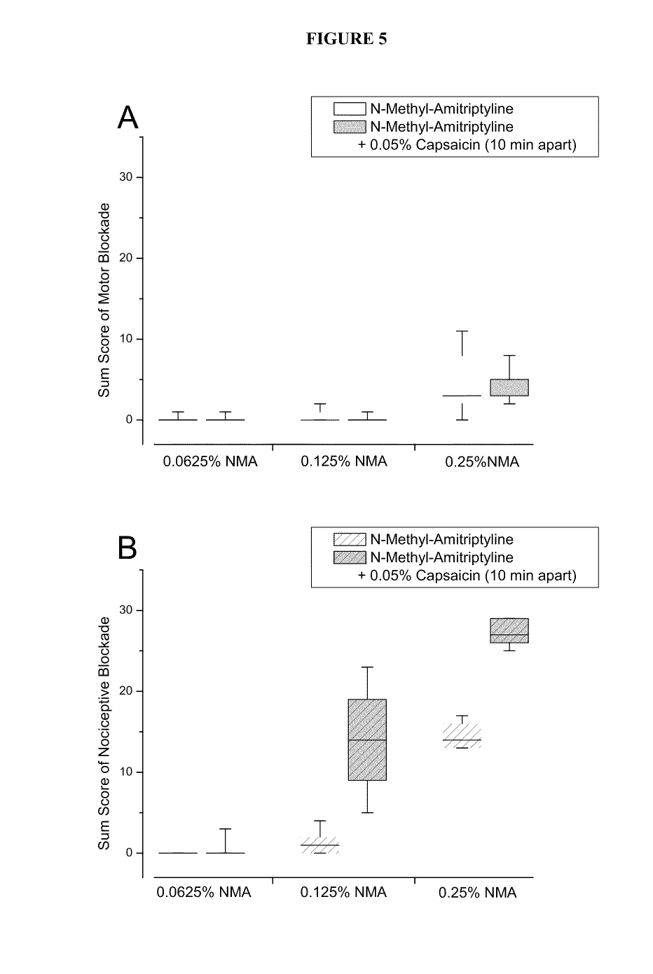

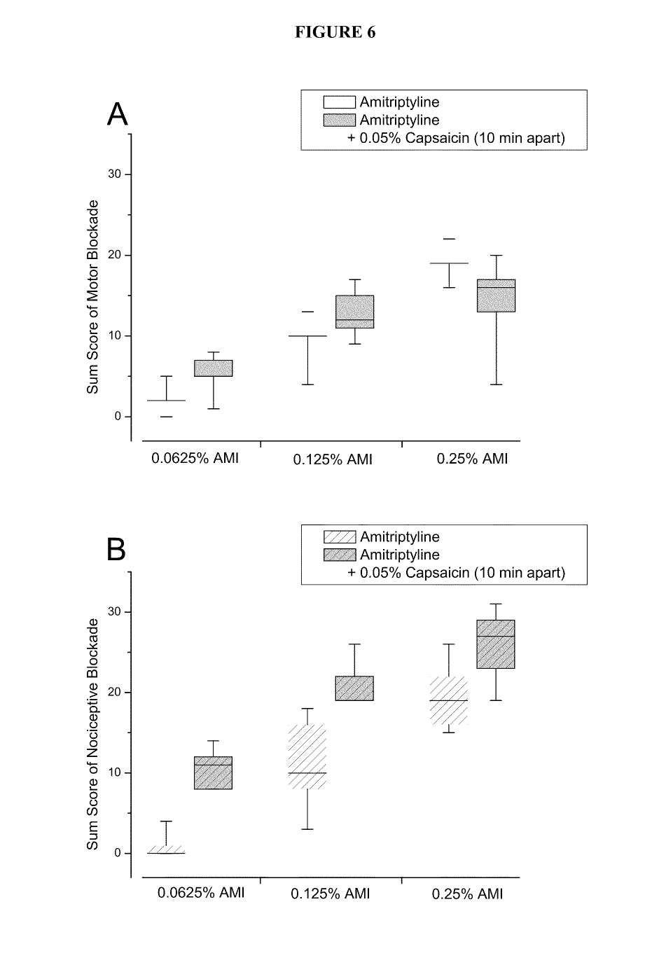

Because sensory/motor separation by local anesthetics is a partially concentration-dependent, dose-response studies were performed for N-methyl amitriptyline and amitriptyline. N-methyl amitriptyline and amitriptyline at concentrations of 0.0625, 0.125, and 0.25% produced dose-dependent and predominantly nociceptive block when the injection was followed by capsaicin (FIGS. 5 and 6, respectively). With amitriptyline at a concentration of 0.25%, the motor block decreased when followed by capsaicin (FIG. 6A).

Injection of capsaicin, normal saline, or vehicle only (solvent of 10% ethanol, 10% Tween®80, and 80% normal saline) caused no detectable block. In addition, injection of the vehicle 10 minutes after the respective local anesthetic produced an immediate and short-lived (2-3 minutes) intensification of both motor and nociceptive block with overall no significant differential block (FIGS. 1-4 and Table 1).

Intragroup comparison demonstrated significant differences among the different dosing groups (Tables 1 and 2). Moreover, the complete recovery time and amount of block data summarized in Table 1 and 2 respectively, show that more hydrophobic drugs such as amitriptyline and bupivacaine (log P value/octanol-buffer coefficient of 4.9 and 3.4, respectively) displayed significantly more differential block than the hydrophilic lidocaine (log P value of 2.3).

The results presented herein demonstrate that permanently charged permeant LAs (e.g., N-methyl amitriptyline) also produce a pronounced differential rat sciatic nerve blockade when co-injected with or followed by an injection of capsaicin. In addition, tertiary amine LAs also provide enhanced and longer-lasting differential block when followed by capsaicin, and more hydrophobic drugs elicit a larger differential block. Results for such permeant LAs demonstrate a nociceptor-predominant sciatic nerve block, but not the nociceptor-selective sciatic nerve block found for the nearly membrane-impermeable LA QX-314 when followed by capsaicin. This result suggests that capsaicin facilitates the entrance of LAs into the nociceptive nerve fibers through TRPV1 channels, but does not interfere substantially with traditional transmembrane crossing of LAs into motor fibers. However, it was also observed that simultaneous application of capsaicin decreased the absolute duration of motor block for the more hydrophobic drugs amitriptyline and bupivacaine. The injection of capsaicin could at least temporarily slightly lower the tissue pH, causing more LA molecules to be positively charged and in turn decreasing the number of LA molecules able to enter the motor nerve fibers. Also, the pKA of lidocaine (7.8) is lower than that of bupivacaine (8.1) and amitriptyline (9.5). Therefore, a significantly higher percentage of lidocaine will be in the uncharged form and therefore available to block motor fibers, in keeping with our results that showed the largest motor block with the drug of lowest pKA (lidocaine).

It also appears that the vehicle itself may play a minor role in the nerve blockade. Injection of the vehicle (10% ethanol, 10% Tween®80, and 80% normal saline) 10 min after bupivacaine or lidocaine led to an intensification of both motor and nociceptive block (FIGS. 3 and 4). This finding is consistent with the known nerve blocking properties of ethanol, and with TRPV1 activation by ethanol.

Finally, given that sodium channels are not the only targets of LAs, the effects demonstrated here might be partly due to differential actions of the various tertiary and quaternary agents on K⁺ channels, Ca²⁺ channels, various ligand-gated channels, second messengers, and substance P neurokinin 1 receptors.

One concern though is that capsaicin causes a severe burning upon injection. However, it was observed that all rats appeared neurobehaviorally normal upon awakening from a short inhalational anesthesia, as indicated by normal grooming, fluid intake, and exploratory behavior, suggesting that the preceding or concomitant use of LAs eliminated this problem. Although no formal toxicity studies have yet been performed, the overall low concentrations of drugs used by Binshtok et al. and in the current study encourage cautious optimism, as does the full return to baseline.

Sciatic nerve block with quaternary ammonium and tertiary amine LAs followed by injection of capsaicin provides a predominantly sensory/nociceptor selective block with a duration that greatly exceeds that produced by the LA alone. Therefore, exploitation of the interaction of TRPV1 receptor agonists and several chemically distinct groups of LAs appears to be a promising path toward regional analgesia without motor block.

### Example 5

### Prolonged Cutaneous Analgesia from Transdermal Penetration of Amitriptyline and Capsaicin

It was hypothesized that the combined application of amitriptyline and capsaicin as a transdermal patch will produce prolonged cutaneous analgesia compared to amitriptyline alone. Male Sprague-Dawley rats (weights 250-300 g) were assigned to five treatment groups (n=6-8). Transdermal patches containing amitriptyline with different concentrations of capsaicin were applied to the rats' shaved backs: 2.5% amitriptyline alone (control group) and in combinations with 0.05%, 0.15%, 1, and 8% capsaicin for 3 hours. Behavioral testing for cutaneous nociception was conducted before and after patch removal, using the cutaneous trunci muscle reflex (CTMR). In addition, skin appearance was assessed to determine irritation by the formulation.

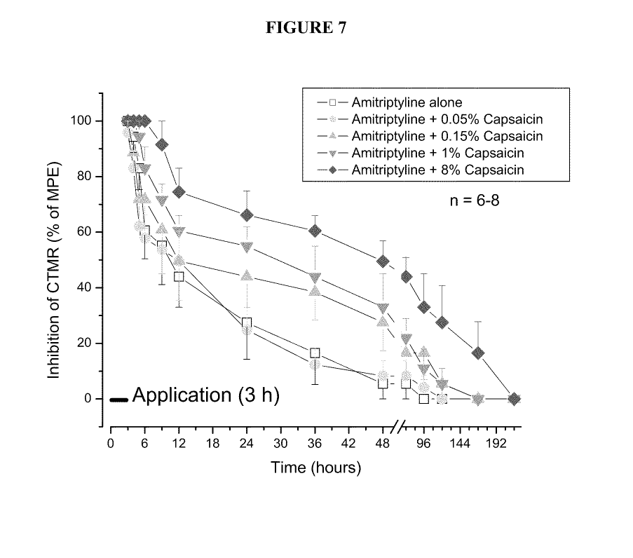

As shown in FIG. 7, a significantly prolonged cutaneous analgesic effect is achieved when amitriptyline is applied in combination with 8% capsaicin. Amitriptyline alone provided a complete block to pinprick for 4.5 hours and the time to full recovery was 96 hours. Amitriptyline with 8% capsaicin produced a complete block to pinprick for 6-9 hours and the time to full recovery was 216 hours (p=0.002). Amytriptyline alone causes toxic effects in skin, while the higher the concentration of capsaicin, the less skin irritation was noted and the combination of amitriptyline 2.5% with capsaicin 8% caused no adverse skin reactions. These data demonstrate that the combined application of amitriptyline and capsaicin results in prolonged cutaneous analgesia compared with amitriptyline alone, suggesting that the activation of the TRPV1 channel by capsaicin facilitates the passage of amitriptyline into nociceptors. The transdermal patch achieves far longer cutaneous analgesia than currently available patch applications. The mechanism that underlies the lesser skin irritation noted when amitriptyline is combined with higher doses of capsaicin compared to amitriptyline alone in unclear but may be related to a counteraction of amitriptyline-induced vasoconstriction.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating pain in a subject the method comprising administering to the subject effective amounts of (i) one or more membrane-permeant local anesthetic agents and (ii) a transient receptor potential cation channel, subfamily V, member 1 (TRPV1) receptor agonist.

2. The method of claim 1, wherein said one or more local anesthetic agents are administered prior to administering said TRPV1 receptor agonist.

3. The method of claim 1, wherein said one or more membrane-permeant local anesthetics is a tricyclic antidepressant.

4. The method of claim 3, wherein said tricyclic antidepressant is selected from the group consisting of amitriptyline, amoxapine, clomipramine, desipramine, doxepin, imipramine, nortriptyline, protriptyline, and trimipramine, or an analog or derivative thereof.

5. The method of claim 1, wherein said one or more membrane-permeant local anesthetic agents are selected from the group consisting of N-methyl amitriptyline, amitriptyline, bupivacaine, lidocaine, N-methyl doxepine, and N-methyl nortriptyline, or an analog or derivative thereof.

6. The method of claim 1, wherein said TRPV1 receptor agonist is an endovanilloid; an endocannabinoid; an anandamide; or a lipoxygenase metabolite of arachidonic acid.

7. The method of claim 6, wherein the endovanilloid is capsaicin.

8. The method of claim 1, wherein the mode of administration is intramuscular, subcutaneous, perineural, neuraxial, or transdermal administration.

9. The method of claim 1, wherein said administration of said one or more membrane-permeant local anesthetics and said administration of said TRPV1 receptor agonist are sequential or substantially simultaneous.

10. The method of claim 9, wherein said administration of said one or more membrane-permeant local anesthetics and said administration of said TRPV1 receptor agonist are separated by a predetermined interval of time.

11. The method of claim 1, wherein said one or more membrane-permeant local anesthetics is N-methyl-amitriptyline and said TRPV1 receptor agonist is capsaicin.

12. The method of claim 11, wherein said N-methyl-amitriptyline and said capsaicin are substantially simultaneously administered by continuous infusion.

13. The method of claim 11, wherein said N-methyl-amitriptyline and said capsaicin are substantially simultaneously administered as a bolus.

14. A composition comprising an analgesic amount of a TRPV1 receptor agonist and an amount of N-methyl-amitriptyline effective to treat pain in a subject administered the composition.

15. A method of treating pain in a subject, the method comprising administering to the subject a therapeutically effective amount of the composition of claim 14.
